(19) United States
(12) Patent Application Publication
(10) Pub. No.: US 2009/0220599 A1
(43) Pub. Date: Sep. 3, 2009

(54) Antifungal formulation and manufacturing method thereof

(75) Inventors: Jih Dar Tsai, Hsinchu City (TW); Lelinda Chang, Changhua County (TW); Shian Jy Jassy Wang, Hsinchu County (TW); Ming Shang Kuo, Taipei City (TW)

Correspondence Address: BACON & THOMAS, PLLC, 625 SLATERS LANE, FOURTH FLOOR, ALEXANDRIA, VA, 22314-1176, US

(73) Assignee: Industrial Technology Research Institute, Hsin Chu (TW)

(21) Appl. No.: 12/382,379

(22) Filed: Mar. 16, 2009

Related U.S. Application Data
(63) Continuation of application No. 10/937,491, filed on Sep. 10, 2004.

(30) Foreign Application Priority Data
Dec. 10, 2003 (TW) 92134826

(51) Int. Cl. A61K 9/30; A61K 9/00; A61K 31/7048

(52) U.S. Cl. 424/475; 424/400; 514/31

(57) ABSTRACT

A new antifungal formulation is provided. The present invention uses a sterol modified with polyethylene glycol (PEG) as a drug carrier. The drug carrier encapsulates Amphotericin B (AmB) by self-assembly to form polymeric micelles. The polymeric micelles can reduce toxicity of Amphotericin B and control release of Amphotericin B. The polymeric micelles of Amphotericin B are used as a new antifungal formulation.

This application is a continuation application of pending U.S. application Ser. No. 10/937,491, filed Sep. 10, 2004, the entirety of which is hereby incorporated by reference.

### BACKGROUND OF THE INVENTION

1. Field of the Invention

The present invention relates to a new antifungal formulation, which uses a sterol modified by polyethylene glycol (PEG) as a drug carrier of Amphotericin B (AmB) in order that the purposes of reducing toxicity and controlled-release of Amphotericin B can be achieved.

2. Description of the Related Art

Medicine research features properties of high technique, high added value, low contamination and low energy cost. The development of new drugs has being trending to new formulations of old drugs for new uses in recent years. The formulation of a drug clinically identified can be modified to change the route of administration of the drug to increase the therapeutic effect, prolong working time and reduce side effects so as to improve added values of the drug and its competition.

Amphotericin B (AmB) is a fermenting product of Streptomyces nodosus, which is a kind of Actinomycetes in the soil. The molecular structure of Amphotericin B is as follows:

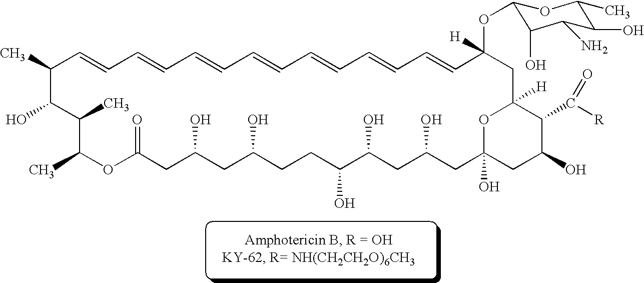

Amphotericin B has an excellent affinity to sterols of biological membranes. Its affinity for ergosterol of fungal cells is ten times better than that for cholesterols of mammal. When Amphotericin B combines with sterols of cell membranes, the cell permeability would be increased, causing lost of potassium ions in the cells, and resulting in the cell broken and apoptosis. It is very effective to treat general fungal infection with Amphotericin B through such mechanism. However, Amphotericin B has a lower affinity for cholesterols though, it is still able to combine with the cell membranes of mammal when entering human bodies, and causing poisoning.

The conventional formulation of Amphotericin B is the drip injection formulation. Amphotericin B deoxycholate (D-Amb) is a primary drug for treatment of fungal infection that threatens human life during last forty years. However, being subject to the tolerance of the patient for this drug and the problem of kidney toxicity caused therefrom, it should be very careful to use this drug. The primary side effects of Amphotericin B include fever, trembling, quickened respiration, muscle pain, nausea, vomiting and headache, etc. The serious side effects could include renal tubule aidosis, lost of capability of concentrating urea, lost of potassium and magnesium ions, and more serious side effect could include myelon inhibition.

Amphotericin B is a concentration-dependent antibiotic. The dosage is subjected to its toxicity. The lipid carrier of Amphotericin B promoted in recent years can reduce general poisoning with symptoms such as trembling, fever, anemia, reduction of blood potassium, nausea and vomiting, etc. It can also reduce kidney's toxicity. The lipid carrier makes Amphotericin B capable of combining the biological degradable lipid with a hydrophilic end and a hydrophobic end. Amphotericin B can be dissolved in water through this dual lipid. Therefore, it would not combine with proteins in blood in a large quantity, while following the lipid to be intake by monocytes of the mononuclear phagocytic system and inflammatory areas of the liver and spleen. Therefore, Amphotericin B can be more selectively to perform the fungicidal interaction. This kind of Amphotericin B formulation can improve therapeutic index and tolerance of the patient for this drug. There are three kinds of Amphotericin B combining with lipid presently existing on the market, including Amphotericin B lipid complex (ABLC), Amphotericin B colloidal dispersion (ABCD) and Liposomal Amphotericin B, L-Amp (AmBisome).

Although the lipid carrier of Amphotericin B existing on the market has less side effects and, conservatively speaking, its clinical effect is equivalent to that of D-AmB, the lipid carrier of Amphotericin B has a high price, which is thirty to sixty-fold higher than the cost of the conventional formulation of Amphotericin B. As a consequence, the lipid carrier of Amphotericin B cannot be widely used.

Accordingly, it is eagerly desirable to develop a new formulation of Amphotericin B, which is effective, capable of reducing toxicity of Amphotericin B and inexpensive.

### SUMMARY OF THE INVENTION

For the purpose of reducing the toxicity of Amphotericin B to mammal's cells, the present invention utilizes the property of Amphotericin B having different affinities for various sterols, choosing a sterol for which Amphotericin B's affinity is between ergosterol and cholesterol as a drug carrier, followed by modifying the sterol with polyethylene glycol, and then using this drug carrier to encapsulate Amphotericin B by self-assembly to form polymeric micelles. When the polymeric micelles of Amphotericin B is delivered into the body, Amphotericin B would combine with ergosterol of the fungus cells' membranes, and not combine with cholesterol of mammal cell's membranes, due to affinity competition. The purposes of controlled-release, reduction of Amphotericin B's toxicity and maintenance of its effectiveness thus can be achieved.

The present invention provides an antifungal formulation, which comprises:

(a) an effective amount of Amphotericin B; and
(b) a sterol modified by polyethylene glycol (PEG);

wherein Amphotericin B is encapsulated by the sterol modified by polyethylene glycol to form polymeric micelles.

The sterol used in the present invention includes ergosterol, cholesterol and stigmasterol, preferably stigmasterol. The aforementioned sterol modified by polyethylene glycol is used as a drug carrier of Amphotericin B, wherein the affinity of Amphotericin B for the sterol modified by polyethylene glycol is smaller than that for ergosterol while larger than that for cholesterol. Therefore, when Amphotericin B encapsulated by the sterol modified with polyethylene glycol is delivered into the mammal's body, Amphotericin B would combine with ergosterol of the fungus cells' membranes, while not combine with cholesterol of mammal cells' membranes, so that the purposes of inhibiting fungal infection by Amphotericin B and reducing damage of Amphotericin B to the mammal are achieved.

Another objective of the present invention is to provide a sterol modified with polyethylene glycol used as the drug carrier of Amphotericin B. The sterol has a formula (I) as follows. The affinity of Amphotericin B for the sterol modified with polyethylene glycol used as the drug carrier is smaller than that for ergosterol, while larger than that for cholesterol.

HO—CH₂—CH₂—(OCH₂CH₂)n-O—X—O-sterol (I)

For the aforementioned formula (I), n is an integer for 10-115, preferably for 12-45; X is a residue group of a compound that both ends have been reacted with the —OH group of polyethylene glycol and sterols. For example, it can be the residue structure of adipoyl chloride that both chloride atoms at the ends thereof have been substituted, or it can be the residue group of carbonate that have been reacted with —OH group.

Still another objective of the present invention is to provide a method for manufacturing an antifungal formulation, which comprises steps of:

(a) selecting a sterol, whose affinity for Amphotericin B is between ergosterol and cholesterol when modified with polyethylene glycol;
(b) modifying the aforementioned sterol with polyethylene glycol in order that polyethylene glycol is covalently attached to —OH group of the sterol directly or indirectly to form a polyethylene glycol-sterol compound; and
(c) mixing the polyethylene glycol-sterol compound of the aforementioned step (b) with Amphotericin B in a proper amount, so that the aforementioned polyethylene glycol-sterol compound encapsulates Amphotericin B by self-assembly to form polymeric micelles.

Yet another objective of the present invention is to provide a method for reducing toxicity of Amphotericin B when being administered into mammals, comprising the step of selecting a compound as a drug carrier, wherein the affinity of said compound for Amphotericin B is smaller than ergosterol and larger than cholesterol.

The antifungal formulation of the present invention prevents Amphotericin B from causing toxicity in mammal by choosing a proper sterol as drug carrier. Because the Amphotericin B has the characteristics that its affinities for various sterols are different, there is competition between drug carrier sterols, ergosterol of fungus cell membranes and cholesterol of mammal cell membranes for combining with Amphotericin B. The affinity of Amphotericin B for the sterol used as the drug carrier in the present invention is smaller than that for ergosterol and larger than that for cholesterol, so that the Amphotericin B of the present antifungal formulation would selectively combine with ergosterol of fungus to develop fungicidal effect without causing toxicity in mammal. In addition, the new antifungal formulation of the present invention can be dissolved in blood by modifying the sterol used as drug carrier with polyethylene glycol, and lowering the probability of the combination of the drug and proteins in blood during the delivery process of the drug. The new antifungal formulation of the present invention can not only reduce toxicity of Amphotericin B but also lower the cost for manufacturing. The present invention has great commercial potential and health care effect.

### BRIEF DESCRIPTION OF DRAWINGS

FIG. 1 shows a curve diagram of A₄₁₅ₙₘ/A₃₅₀ₙₘ vs. molar ratio of sterol/AmB.

FIG. 2 shows a curve diagram of A₄₁₅ₙₘ/A₃₅₀ₙₘ vs. molar ratio of sterol-PEG600/AmB.

### DETAILED DESCRIPTION OF THE INVENTION

The present invention provides an antifungal formulation including an effective amount of Amphotericin B and a sterol modified with polyethylene glycol (PEG). The aforementioned Amphotericin B is encapsulated by the sterol modified with polyethylene glycol to form polymeric micelles. The size of the polymeric micelles is in the range of 70-300 nm. The polymeric micelles can be further processed to various forms, such as injection, tablet and semisolid, etc.

The aforementioned sterol modified with polyethylene glycol (PEG) has a structure of the following formula (I), which is used as a drug carrier of Amphotericin B,

HO—CH₂—CH₂—(OCH₂CH₂)n-O—X—O-sterol (I),

Wherein X is a residue group of a compound that both ends have been reacted with the —OH group of polyethylene glycol and sterols. For example, it can be the residue structure of adipoyl chloride that both chloride atoms at ends thereof have been substituted or it can be the residue group of carbonate that have been reacted with —OH group. The molecular weight of polyethylene glycol used for modifying the sterol is in the range of 600-5000, and n in the formula (I) is an integer of 10-115, preferably 12-45.

The sterol of the compound of formula (I) includes ergosterol, cholesterol and stigmasterol. The compound of formula (I) includes HO—CH₂—CH₂—(OCH₂CH₂)n-O—X—O-ergosterol, HO—CH₂—CH₂—(OCH₂CH₂)n-O—X—O-cholesterol or HO—CH₂—CH₂—(OCH₂CH₂)n-O—X—O-stigmasterol, preferably HO—CH₂—CH₂—(OCH₂CH₂)n-O—X—O-stigmasterol, and most preferably is a compound of the following formula (II), wherein n is an integer of 10-115, preferably is 12-45.

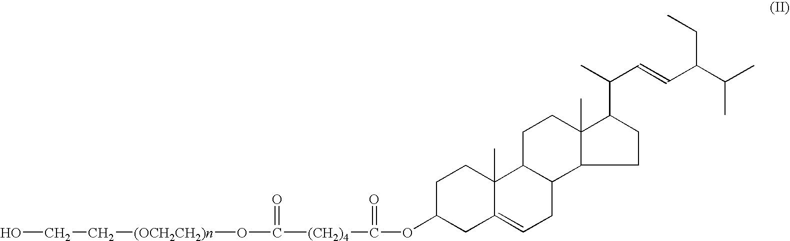

The affinity of Amphotericin B for the sterol modified with the aforementioned ethylene glycol is smaller than that for ergosterol and larger than that for cholesterol. The present invention utilizes the property of excellent affinity of Amphotericin B for the sterol to encapsulate Amphotericin B with the sterol by affinity self-assembly to form polymeric micelles. After Amphotericin B encapsulated by the sterol modified with polyethylene glycol delivered into the mammal, it can combine with ergosterol of fungus cell membranes, while not combine with cholesterol of mammal cell membranes. The purposes of inhibiting fungal infection with Amphotericin B and reducing damage of Amphotericin B for the mammal are achieved.

The purpose of modifying the sterol with polyethylene glycol is to provide polymeric micelles formed of Amphotericin B encapsulated by the polyethylene glycol-sterol carrier with a hydrophilic end, such that the polymeric micelles of Amphotericin B can be dissolved in water and will not combine with proteins in blood in a large quantity. As a consequence, the kidney poisoning can be reduced and the polymeric micelles of Amphotericin B can follow lipid to be intake by monocyte.

The polymeric micelles of Amphotericin B encapsulated by the sterol modified with polyethylene glycol can be dispersed in a proper solvent for preservation. The proper solvent is a co-solvent including methanol/acetone, methanol/acetonitrile and ethanol/acetone. The mixture ratio of the aforementioned co-solvent is in the range of 1/1 to 1/5, preferably 1/2.

The present invention provides a method for manufacturing an antifungal formulation, which includes the steps of: (a) selecting a sterol, whose affinity for Amphotericin B is between ergosterol and cholesterol when modified with polyethylene glycol; (b) modifying the aforementioned sterol with polyethylene glycol in order that polyethylene glycol is covalently attached to —OH group of the sterol directly or indirectly to form a polyethylene glycol-sterol compound; and (c) mixing the polyethylene glycol-sterol compound of the aforementioned step (b) with Amphotericin B in a proper amount, so that the aforementioned polyethylene glycol-sterol compound encapsulates Amphotericin B by self-assembly to form polymeric micelles.

The polymeric micelles of the aforementioned step (c) are dispersed in a proper solvent for preservation. The proper solvent is a co-solvent including methanol/acetone, methanol/acetonitrile and ethanol/acetone. The mixture ratio of the co-solvent is in the range of 1/1 to 1/5, preferably 1/2.

The following examples are only used to illustrate the present invention, not intended to limit the scope thereof. Many modifications of the examples can be made without departing from the spirit of the present invention, and shall be included in the protection scope of claims of the invention.

### Example I

### Preparation for Sterol-PEG600

### (A) Preparation for Cholesterol-PEG600

Adding 11.6 g (0.03 mol) cholesterol and 4 g (0.04 mol) triethylamine to 100 ml dry toluene to prepare solution A; adding 7.6 g (0.04 mol) adipoyl chloride to 100 ml dry toluene to prepare solution B; slowly adding solution B to solution A to prepare a mixture at a flow rate of 3 ml/min, and stirring for 1.5 hours at a temperature of 12° C.; then centrifugalizing the mixture and filtering it to obtain filtrate, keeping the filtrate for subsequent use; next, adding 36 g (0.06 mol) polyethylene glycol 600 (PEG600) and 6 g (0.06 mole) triethylamine to 100 ml dry toluene, and then adding the mixture to the aforementioned filtrate, stirring for 1.5 hours, to prepare another mixture. Afterward, washing this mixture with 80% (w/w) NaCl aqueous solution four times (50 ml per time), taking a toluene layer out of this mixture and vaporizing toluene in the vaporizer under reduced pressure to leave residue; using 150 ml methanol to wash the residue and filtering insoluble materials therein to leave filtrate; thereafter, extracting the filtrate with ether in an amount equal to the filtrate to obtain extract, then vaporizing ether in the extract. 0.153 g cholesterol-PEG 600 is thus prepared.

### (B) Preparation for Stigmasterol-PEG600

Adding 2.478 g stigmasterol and 1.6 triethylamine to 40 ml dry toluene to prepare solution A; adding 2.42 g adipoyl chloride to 40 ml dry toluene to prepare solution B; slowly adding solution B to solution A at a flow rate of 3 ml/min to prepare a mixture, stirring for 1.5 hours at a temperature of 12° C.; then centrifugalizing the mixture and filtering it to obtain filtrate, keeping the filtrate for subsequent use; next, adding 14.4 g polyethylene glycol 600 (PEG600) and 2.4 triethylamine to 40 ml dry toluene to prepare a mixture, and then adding the mixture to the aforementioned filtrate, stirring for 1.5 hours, to prepare another mixture. Afterward, washing this mixture with 80% (w/w) NaCl aqueous solution four times (25 ml per time), taking a toluene layer out from this mixture and vaporizing toluene in the vaporizer under reduced pressure to leave residue; using 50 ml methanol to wash the residue and filtering insoluble materials therein to obtain filtrate; thereafter, extracting the filtrate with ether in an amount equal to the filtrate to obtain extract, then vaporizing ether in the extract. 1.53 g stigmasterol-PEG600 is thus prepared.

(C) Preparation for ergosterol-PEG600

Adding 1.19 g ergosterol and 0.4 g triethylamine to 10 ml dry toluene to prepare solution A; adding 0.76 g adipoyl chloride to 10 ml dry toluene to prepare solution B; slowly adding solution B to solution A at a flow rate of 3 ml/min to prepare a mixture, stirring for 1.5 hours at a temperature of 12° C.; then centrifugalizing the mixture and filtering it to obtain filtrate, keeping the filtrate for subsequent use; next, adding 3.6 g polyethylene glycol 600 (PEG600) and 0.6 g triethylamine to 10 ml dry toluene to prepare a mixture, and then adding the mixture to the aforementioned filtrate to prepare another mixture, stirring for 1.5 hours. Afterward, washing this mixture with 80% (w/w) NaCl aqueous solution four times (20 ml per time), taking a toluene layer out of this mixture and vaporizing toluene in the vaporizer to leave residue; using 250 ml methanol to wash the residue and filtering insoluble materials therein to leave filtrate; thereafter, extracting the filtrate with ether in an amount equal to the filtrate, then vaporizing ether in the extract. 0.52 g ergosterol-PEG600 is thus prepared.

### Example II

### Comparison of Affinities of Sterol/AmB and Sterol-PEG/AmB

### (A) Measurement of UV-Vis Absorbance Ratio of Sterol/AmB (Instrument Hitachi U-3300):

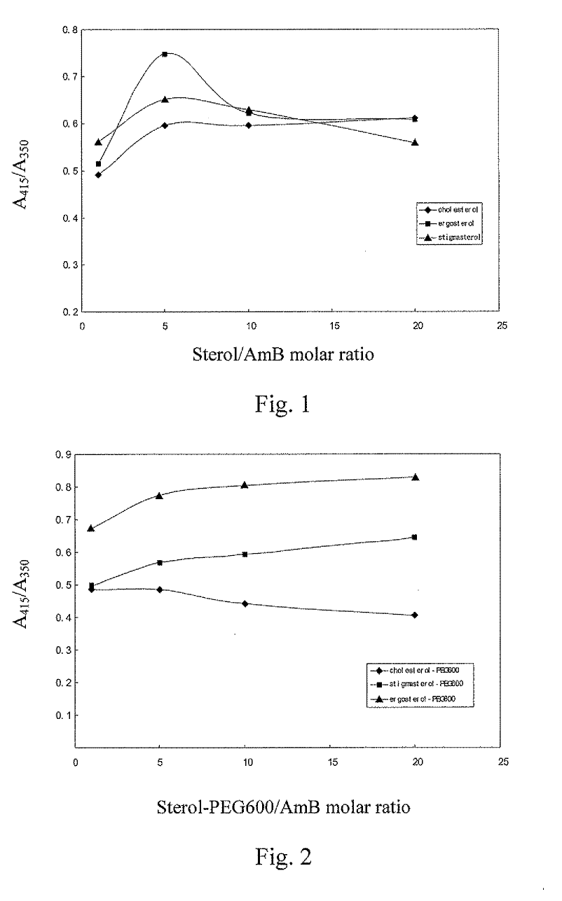

(1) Adding 23.1 mg Amphotericin B (AmB) to 5 ml dimethyl sulfoxide (DMSO), and then adding in 495 ml ionized water to prepare a mixture, and taking 10 ml volume out of the mixture; diluting the 10 volume with 30 ml 1% dimethyl sulfoxide (DMSO) solution to prepare 12.5 μM Amphotericin B (AmB) aqueous solution.
(2) Adding 13 g n-propanol to 187 ml ionized water to prepare 6.5% n-propanol aqueous solution.
(3) Adding 28.9 mg cholesterol to 19.5 g n-propanol, shaking them to completely dissolve cholesterol in n-propanol, and then adding in 280.5 gram ionized water to prepare 250 μM cholesterol aqueous solution.
(4) Adding 30.9 g stigmasterol to 19.5 g n-propanol, shaking them to completely dissolve stigmasterol in n-propanol, and then adding in 280.5 gram ionized water to prepare 250 μM stigmasterol aqueous solution.
(5) Adding 29.7 mg ergosterol to 19.5 g n-propanol, shaking them to completely dissolve ergosterol in n-propanol, and then adding in 280.5 gram ionized water to prepare 250 μM ergosterol aqueous solution.
(6) Mixing 10 ml 12.5 μM AmB aqueous solution with 10 ml 250 μM cholesterol aqueous solution. The molar ratio of AmB/cholesterol is 1/20. The UV-Vis absorbance ratio A₄₁₅ₙₘ/A₃₅₀ₙₘ of AmB/cholesterol is 0.61 (scanning from 300 nm to 450 nm).
(7) Mixing 10 ml 12.5 μM AmB aqueous solution, 5 ml 6.5% n-propanol aqueous solution and 5 ml 250 μM cholesterol aqueous solution. The molar ratio of AmB/cholesterol is 1/10. The UV-Vis absorbance ratio A₄₁₅ₙₘ/A₃₅₀ₙₘ of AmB/cholesterol is 0.59.
(8) Mixing 10 ml 12.5 μM AmB aqueous solution, 7.5 ml 6.5% n-propanol aqueous solution and 2.5 ml 250 μM cholesterol aqueous solution. The molar ratio of AmB/cholesterol is 1/5. The UV-Vis absorbance ratio A₄₁₅ₙₘ/A₃₅₀ₙₘ of AmB/cholesterol is 0.60.
(9) Mixing 10 ml 12.5 μM AmB aqueous solution, 9.5 ml 6.5% n-propanol aqueous solution and 0.5 ml 250 μM cholesterol aqueous solution. The molar ratio of AmB/cholesterol is 1/1. The UV-Vis absorbance ratio A₄₁₅ₙₘ/A₃₅₀ₙₘ of AmB/cholesterol is 0.49.
(10) Measuring absorbance ratio of stigmasterol/AmB and ergosterol/AmB by the procedures with the same, the result is shown in FIG. 1.

### (B) Measuring UV-Vis Absorbance Ratio of Sterol-PEG/AmB (Instrument Hitachi U-3300):

(1) Adding 23.1 mg AmB aqueous solution to 5 ml dimethyl sulfoxide (DMSO), and then adding in 495 ml ionized water to prepare a mixture. Taking 10 ml volume out of the mixture, and diluting the 10 ml volume with 30 ml 1% dimethyl sulfoxide (DMSO) aqueous solution to prepare 12.5 μM AmB aqueous solution.
(2) Adding 13 g n-propanol to 187 ml ionized water to prepare 6.5% n-propanol aqueous solution.
(3) Adding 24.4 mg cholesterol-PEG600 to 6.5 g n-propanol, shaking them to completely dissolve cholesterol-PEG600 in n-propanol, and then adding in 93.5 g ionized water to prepare 250 μM cholesterol-PEG600 aqueous solution.
(4) Adding 25.0 g stigmasterol-PEG600 to 6.5 g n-propanol, shaking them to completely dissolve stigmasterol-PEG600 in n-propanol, and then adding in 93.5 g ionized water to prepare 250 μM stigmasterol-PEG600 aqueous solution.
(5) Adding 24.6 mg ergosterol-PEG600 to 6.5 g n-propanol, shaking them to completely dissolve ergosterol-PEG600 in n-propanol, and then adding in 93.5 g ionized water to prepare 250 μM ergosterol-PEG600 aqueous solution.
(6) Mixing 10 ml 12.5 μM AmB and 10 ml 250 μM cholesterol-PEG600, the molar ratio of AmB/cholesterol-PEG600 is 1/20. The UV-Vis absorbance ratio A₄₁₅ₙₘ/A₃₅₀ₙₘ of AmB/cholesterol is 0.40 (scanning from 300 nm to 450 nm).
(7) Mixing 10 ml 12.5 μM AmB, 5 ml 6.5% n-propanol and 5 ml 250 μM cholesterol-PEG600. The molar ratio of AmB/cholesterol-PEG600 is 1/10. The UV-Vis absorbance ratio of A₄₁₅ₙₘ/A₃₅₀ₙₘ of AmB/cholesterol-PEG600 is 0.44.
(8) Mixing 10 ml 12.5 μM AmB, 7.5 ml 6.5% n-propanol and 2.5 ml 250 μM cholesterol-PEG600. The molar ratio of AmB/cholesterol-PEG600 is 1/5. The UV-Vis absorbance ratio of A₄₁₅ₙₘ/A₃₅₀ₙₘ of AmB/cholesterol-PEG600 is 0.48.
(9) Mixing 10 ml 12.5 μM AmB, 9.5 ml 6.5% n-propanol and 0.5 ml 250 μM cholesterol-PEG600. The molar ratio of AmB/cholesterol-PEG600 is 1/1. The UV-Vis absorbance ratio of A₄₁₅ₙₘ/A₃₅₀ₙₘ of AmB/cholesterol-PEG600 is 0.48.
(10) Measuring absorbance ratio of stigmasterol-PEG600/AmB and ergosterol-PEG600/AmB by the procedures with the same, the result is shown in FIG. 2.

Using UV-Vis spectroscopy to measure UV-Vis absorbance ratios of cholesterol-PEG600, stigmasterol-PEG600 and ergosterol-PEG600 prepared in Example I to AmB and UV-Vis absorbance ratios of cholesterol, stigmasterol and ergosterol to AmB. The results are as shown in FIG. 1 and FIG. 2, in which FIG. 1 is a curve diagram of A₄₁₅ₙₘ/A₃₅₀ₙₘ vs. sterol/AmB and FIG. 2 is a curve diagram of A₄₁₅ₙₘ/A₃₅₀ₙₘ vs. sterol-PEG600/AmB.

In view of FIG. 1, it appears that the UV-Vis absorbance ratios (A₄₁₅ₙₘ/A₃₅₀ₙₘ) vary with molar ratios (from 1:1 to 20:1) of cholesterol/AmB, stigmasterol/AmB and ergosterol/AmB respectively. When molar ratio of sterol/AmB is 1/5, the UV-Vis absorbance ratios of the sterol/AmB show the most remarkable difference. The affinity ordering of these three sterols to AmB is cholesterol<stigmasterol<ergosterol. In view of FIG. 2, for any mixing molar ratios of cholesterol-PEG600/AmB, stigmasterol-PEG600 and ergosterol-PEG600, the variation of the UV-Vis absorbance ratios A₄₁₅ₙₘ/A₃₅₀ₙₘ is insignificant, and the affinity ordering of sterol-PEG to AmB is ergosterol-PEG600>stigmasterol-PEG600>cholesterol-PEG600. The above results show that the differences among the affinities of AmB to the sterols modified by polyethylene glycol are more significantly distinguished than that to the sterol without modification.

### Example III

### Preparation for Polymeric Micelles of Stigmasterol-PEG/AmB

Mixing 125 mg the aforementioned prepared stigmasterol-PEG600 and 30 mg AmB, and then being dissolved in a 30 ml co-solvent to prepare a solution. The pH value of the solution is adjusted to 3 with 0.1 N HCl aqueous solution. Then, the solution is heated to 50° C. and sonic shaking for about 10 minutes. Thereafter, the solution is added to 40 ml ionized water, in which 125 mg surfactant Pluronic F68 is previously dissolved, to prepare a mixture. The mixture is stirred with a magnetic bar for 30 minutes. Subsequently, the mixture is concentrated to 10 ml in a vaporizer at a 55° C. water bath to obtain yellow suspension, that is polymeric micelles of AmB encapsulated by stigmasterol-PEG600 and the concentration is 3 mg/ml.

The co-solvents used in the Example III include methanol/acetone, methanol/acetonitrile and ethanol/acetone. When using methanol/acetone (10 ml/20 ml) as a co-solvent, the measured particle size of the polymeric micelles is about 109 nm (CV=112%); when using methanol/acetonitrile (10 ml/20 ml) as a co-solvent, the measured particle size of the polymeric micelles is about 120 nm (CV=124%); when using ethanol/acetone (10 ml/20 ml) as a co-solvent, the measured particle size of the polymeric micelles is about 72 nm (CV=81%).

When the polymeric micelles of stigmasterol-PEG600/AmB are delivered into the body, they are supposed to release AmB if encountering with fungui cells containing ergosterol, in view of the result of the aforementioned Example II, AmB would combine with ergosterol due to that AmB has larger affinity for ergosterol. The fungui cells are thus killed. While the polymeric micelles of stigmasterol-PEG600/AmB are delivered into the body, AmB would not be released from the polymeric micelles if encountering with normal human cells having cholesterol. The controlled-release of AmB of the polymeric micelles in the body is obtained by way of affinity differences of AmB with different sterols.

1-25. (canceled)

26. A method for reducing toxicity of Amphotericin B when administered into mammals, comprising the steps of:
administering an antifungal formulation, comprising (a) an effective amount of Amphotericin B, and (b) a sterol modified by polyethylene glycol having a structure of formula (I) to said mammals, in which, Amphotericin B is encapsulated by said sterol modified by polyethylene glycol to form polymeric micelles:
HO—CH₂—CH₂—(OCH₂CH₂)ₙ—O—X—O-sterol (I),
wherein X is a residue group of a compound wherein both ends have been reacted with the —OH group of polyethylene glycol and sterols, n is an integer of 10-115; and
wherein the affinity of Amphotericin B for said sterol modified by polyethylene glycol is smaller than that for ergosterol and larger than that for cholesterol so that Amphotericin B is able to combine with ergosterol of the membrane of fungus cells, not able to combine with cholesterol of the membrane of said mammals after said Amphotericin B enters said mammals, thereby reducing toxicity of Amphotericin B and effectively releasing Amphotericin B.

27. The method of claim 26, wherein the size of the polymeric micelle is in the range of 70-300 nm.

28. The method of claim 26, wherein the sterol modified by the polyethylene glycol encapsulates Amphotericin B by affinity self-assembly to form the polymeric micelles.

29. The method of claim 26, wherein the sterol modified by polyethylene glycol is a drug carrier of Amphotericin B.

30. The method of claim 26, wherein the molecular weight of polyethylene glycol is in the range of 600-5000.

31. The method of claim 30, wherein the molecular weight of polyethylene glycol is 600.

32. The method of claim 26, wherein the antifungal formulation is administered in the form of an injection, tablet, or semisolid.

33. The method of claim 26, wherein the polymeric micelles are dispersed in a solvent for preservation.

34. The method of claim 33, wherein the solvent is a co-solvent with a mixture ratio of 1/1 to 1/5, including methanol/acetone, methanol/acetonitrile and ethanol/acetone.

35. The method of claim 34, wherein the mixture ratio of the co-solvent is 1/2.

36. The method of claim 26, wherein the sterol modified by polyethylene glycol includes HO—CH₂—CH₂—(OCH₂CH₂)ₙ—O—X—O-ergosterol, HO—CH₂—CH₂—(OCH₂CH₂)ₙ—O—X—O-cholesterol or HO—CH₂—CH₂—(OCH₂CH₂)ₙ—O—X—O-stigmasterol; wherein X is a residue group of a compound that both ends have been reacted with the —OH group of polyethylene glycol and sterols, n is an integer of 10-115.

37. The method of claim 36, wherein the sterol modified by polyethylene glycol is HO—CH₂—CH₂—(OCH₂CH₂)n-O—X—O-stigmasterol; wherein X is a residue group of a compound that both ends have been reacted with the —OH group of polyethylene glycol and sterols, n is an integer of 10-115.

38. The method of claim 37, wherein the sterol modified by polyethylene glycol is a compound of the following formula (II): 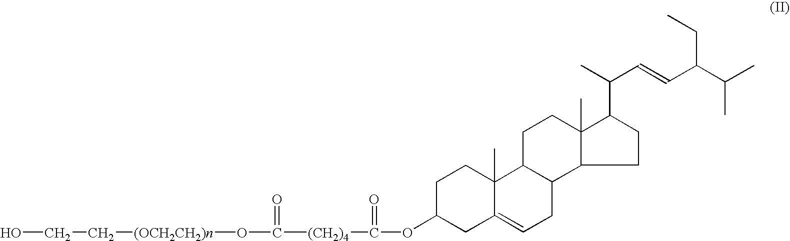
wherein n is an integer of 10-115.

39. The method of claim 26, wherein n of the formula (I) is an integer of 12-45.

40. The method of claim 26, wherein X of formula I is adipoyl chloride wherein both chlorides at the end of said adipoyl chloride have been reacted with the —OH group of polyethylene glycol and sterols.
